(19) United States
(12) Patent Application Publication
(10) Pub. No.: US 2025/0152394 A2
(43) Pub. Date: May 15, 2025

(54) JOINT DEVICE

(71) Applicant: Otto Bock Healthcare Products GmbH, Wien (AT)

(72) Inventors: Harald SIMA, Herzogenburg (AT); Roland AUBERGER, Wien (">)

(73) Assignee: Otto Bock Healthcare Products GmbH, Wien (AT)

(21) Appl. No.: 16/642,334

(22) PCT Filed: Aug. 28, 2018
(86) PCT No.: PCT/EP2018/073131

§ 371 Date: Feb. 26, 2020

(30) Foreign Application Priority Data
Sep. 14, 2017 (DE) 10 2017 121 343.0

(51) Int. Cl. A61F 5/01

(52) U.S. Cl. CPC A61F 5/0125; A61F 2005/0134; A61F 2005/0137; A61F 2005/0146; A61F 2005/0151; A61F 2005/0155; A61F 2005/0165

(57) ABSTRACT

A joint device of an orthosis with an upper part and with a lower part that is arranged on the upper part in an articulated manner, with a first fastening device for securing the upper part to a patient and a second fastening device for securing the lower part to a limb, wherein the joint device connects the upper part to the lower part in an articulated manner and has an upper part binding and a lower part binding via which the upper part and the lower part can be secured to the fastening devices, wherein the joint device has at least four degrees of freedom.

The invention relates to a joint device of an orthosis with an upper part and with a lower part arranged on the latter in an articulated manner, with a first fastening device for securing the upper part to a patient and a second fastening device for securing the lower part to a limb of a patient, wherein the joint device connects the upper part to the lower part in an articulated manner and has an upper-part connection and a lower-part connection, via which the upper part and the lower part can be secured to the fastening devices.

Orthoses are fitted in place on the trunk and/or limb of a patient and generally have rails or shells with devices for securing the respective rails or shells to the trunk or the limb. The rails or shells are connected to each other via a joint device, such that the orthosis can be arranged on the patient in a manner engaging over the joints. By means of an orthosis, movements can be performed, pivot angles about a joint axis can be limited, pivoting movements can be prevented, or an orientation of limbs relative to each other can be assisted or fixed. In addition, orthoses can be provided with damper elements and/or spring elements or drives in order, respectively, to damp pivoting movements about the respective joint axis or to effect or support such movements. The damper and/or the drive can be provided with a controller, such that modified damping or modified movement support in the flexion direction or extension direction can be made available, depending on loads, pivot angles, speeds or accelerations, which are detected via sensors and are evaluated in a control device.

The damper devices can be configured as hydraulic damper devices, pneumatic damper devices or a combination of hydraulic and pneumatic damper devices. By means of magnetorheological substances, dampers can likewise change the resistance to a movement by changing an applied magnetic field. In addition to purely passive damper devices, there are orthoses having active drives in which a displacement of the upper part relative to the lower part in a displacement direction is effected via an electric motor, for example.

Orthoses can engage over just a single joint, for example a hip joint, a knee joint or an ankle joint. It is alternatively possible and provided that the orthosis engages over several joints, for example both the hip joint and the knee joint and the ankle joint, or only a hip joint and a knee joint. In an orthosis that engages over one or more natural joints, the proximal component of the orthosis is in each case the upper part, and the distal component is in each case the lower part. In a knee-ankle-foot orthosis, therefore, a lower-leg rail is the lower part in relation to the portion engaging over the knee, since the lower-leg rail is located distally with respect to an upper-leg rail. With respect to a foot rail or foot shell, the lower-leg rail is then an upper part, while the foot rail or foot shell, which is connected to the lower-leg rail via an ankle orthosis, forms the lower part. In an orthosis engaging over the hip joint, the part arranged on the trunk or pelvis of the patient is the upper part, while the element of the orthosis fastened to the upper leg forms the lower part.

As regards the arrangement of actuators, i.e. drives and/or dampers, on an orthosis, the problem is that, on account of different anatomies, manufacturing tolerances and soft-tissue movements in the region of the places where the orthosis is fitted on the body of the patient, there is a possibility of alignment errors between the orthosis axis and the anatomical joint axis. In more complex joints in particular, such as the hip joint or the shoulder joint, but also the knee joint and ankle joint, it can be difficult, with a rigid design of the joint device between the upper part and the lower part, to achieve sufficient congruence between the anatomical axes, or the anatomical axis, and the axis or axes of the orthosis.

The object of the present invention is to make available a joint device for an orthosis allowing the joint device to be positioned near the natural joint and always allowing the orthosis to be fitted close to the body of the patient.

According to the invention, this object is achieved by a joint device having the features of the main claim. Advantageous embodiments and developments of the invention are disclosed in the dependent claims, the description and the figures.

The joint device of an orthosis with an upper part and with a lower part arranged in an articulated manner, with a first fastening device for securing the upper part to a patient and a second fastening device for securing the lower part to a limb, wherein the joint device connects the upper part to the lower part in an articulated manner and has an upper-part connection and a lower-part connection, via which the upper part and the lower part can be secured to the fastening devices and the joint device, makes provision for the joint device to have at least four degrees of freedom. On account of its at least four degrees of freedom, the joint device as such is kinematically indeterminate, such that the orthosis becomes kinematically determinate as a system, of which a component is the joint device, only when placed onto the patient or onto the user. The joint device as such is preferably configured as a prefabricated module which can be connected with the upper-part connection and the lower-part connection to the respective fastening device, for example by screwing or riveting. The respective fastening device can be configured as a shell, which engages at least partially around the respective body part, or as a belt or strap arrangement, in order to secure the orthosis overall to the patient. In the case of an orthosis engaging over the hip joint, the first fastening device can be configured to secure the upper part to a patient, for example a pelvic strap or a pelvic shell, while the fastening device for securing the lower part can be configured as an upper-leg shell or upper-leg strap. In the use as a shoulder orthosis, the upper part is secured to a fastening device which is placed on the trunk, while the fastening device for the lower part is arranged on the upper arm and can be configured as a shell or cuff which, by way of a strap or a hook-and-loop fastener, can be placed completely around the upper arm.

By virtue of the configuration of the joint device with at least four degrees of freedom, it is possible to configure the joint device such that an automatic positioning of the components relative to each other is permitted within certain movement limits, as a result of which the resulting rotation axes of the joint device always run through the physiological rotation point of the natural joint, thereby avoiding constraints, movement limitations, or joint loads resulting from alignment errors. Thus, it is no longer necessary to exactly determine the anatomical rotation point and to align the orientation of the rotation axes of the joint device precisely with respect to the anatomical rotation point once the latter has been found. Instead, after the orthosis has been fitted in place, the joint device automatically aligns itself correctly to the physiologically correct rotation point, within the predefined limits of the mobility. By virtue of the configuration of the joint device with upper-part connection and lower-part connection, it is possible to separate the fastening devices from the joint device, such that fitting an orthosis in place is made easier.

The joint device preferably has three rotational degrees of freedom, in order also to be able to bridge ball joints, such as the hip joint or the shoulder joint, and to support them in the context of treatment by orthosis.

The joint device can have at least one translational degree of freedom, such that a displacement possibility is permitted in at least one direction. In a development of the invention, the joint device can have at least two freedom, such translational degrees of that a displacement possibility is permitted in two different directions. By means of the displaceability along at least one translational degree of freedom, it is possible to prevent a situation where a fastening device is shifted in an arrangement of the respective joint device on the body part. For example, in an embodiment of the joint device as part of an orthosis engaging over the hip joint, a translational degree of freedom normal to the sagittal plane can be free, but can be limited by end stops. With the orthosis fitted in place, this translational degree of freedom brings about an alignment of the functional rotation point of the joint unit with the anatomical hip joint. In addition, a translational degree of freedom normal to the horizontal plane can be provided, such that the lower-part connection can be longitudinally displaceable along the longitudinal extent of the upper leg.

In a variant of the invention, provision is made that the rotation axes of at least two rotational degrees of freedom intersect each other, such that rotations about one rotation axis have no effects on movements about the other rotation axis.

In order also to be able to support joints whose rotation axes or rotation points lie within the body, it is advantageous if the pivot axis of at least one rotational degree of freedom lies outside the joint device, such that, for a hip joint for example, an abduction and adduction of the leg can also be permitted about a pivot axis perpendicular to the frontal plane. For this purpose, an elongate hole guide is preferably provided in the joint device, said elongate hole guide having a curved elongate hole whose contour has a portion of a circular orbit, wherein the midpoint of the circular orbit lies outside the mechanical components of the joint device. The midpoint of this curved path preferably lies in the rotation point or near the rotation point of the natural joint on which the joint device is arranged. By shortening the elongate hole, e.g. by inserting elastic stop elements, the movement range in this degree of freedom can be reduced. Likewise, the behavior of the joint in this rotational or translational degree of freedom can be influenced by insertion of rigid or elastic bands between the element guided in the elongate holes and the end points of the elongate holes. Stops can be created with rigid bands, wherein the stop position is defined by the length of the bands. Elastic bands permit implementation of an elastic behavior about this degree of freedom, wherein the neutral position is determined by the length of the bands and the stiffness is determined by the elasticity properties of the bands. A progressive behavior can be achieved by the parallel use of several bands of different length or stiffness. In the case of an orthosis of the lower extremity, this would be the hip joint; in the case of an orthosis of the upper extremity, this would be the shoulder joint.

At least one elastic buffer element or spring element is assigned to at least one degree of freedom, in order to achieve a stabilization of the joint device with respect to the degree of freedom in question. In addition, a limitation of the displaceability about this degree of freedom or in this degree of freedom is achieved via the spring or buffer element. In addition, besides providing a limitation of the displacement path or displacement angle, the spring or buffer element can additionally have the effect that the movement in or about this degree of freedom is provided with a resistance, for example a frictional resistance, so as to be able to adapt the joint device and the behavior of the joint device to the individual requirements.

The spring or buffer element or the spring or buffer elements of one rotational degree of freedom can be formed by elastomer elements arranged parallel to the rotation axis, such that the torsional stiffness can be adjusted easily and steplessly by a change of the length of the elastomer elements, which are preferably configured in a cord shape. In addition, the desired elastic behavior can be set by the shape of the cross section of the spring or buffer elements. Thus, for example, a progressive elastic behavior can be set by an increasing cross section in the displacement direction. It is also possible to set the corresponding elastic properties by pairing of materials. A hard stop can be realized by use of very stiff spring or buffer elements. The neutral position in the case of elastic behavior, or the stop positions, can be influenced by the diameter of the spring or buffer elements that are used. This is of interest particularly when using very stiff elements, since in this way the degree of freedom can be blocked in a defined position by the suitably selected diameters of the spring or buffer elements.

In a development of the invention, provision is made that an actuator is assigned to at least one rotational degree of freedom, which actuator can be configured as a drive or damper. Purely passive dampers can be hydraulic dampers, pneumatic dampers or magnetorheological dampers, wherein combinations of the modes of action are also possible. Other mechanical brakes can also be arranged. In addition, active actuators in the form of drives, in particular motor drives, can be provided in order to support movements and to apply forces or moments for supporting the movement performed by the patient. If the applied forces of the movement introduced by the patient are in opposite directions, the drive forms an active resistance device and is then likewise a damper. Thus, drives can be used both for support and as dampers. Sensors arranged on the orthosis or on the patient can be assigned to the dampers. Information concerning active forces, moments, speeds, accelerations, relative positions or settings of limbs or of orthosis components in space is detected via the sensors, forwarded to a control device and evaluated. On the basis of the evaluated sensor data, the control device, by way of an adjustment device driven by motor, can modify the properties of a passive damper, for example it can increase or reduce a damping. In the case of drives, a corresponding activation of the respective drive can be effected on the basis of the evaluated sensor data. In order to effect a drive of the orthosis or also only an adjustment of a damper device, at least one energy storage means is assigned to the joint device.

The actuator, i.e. the damper or drive, can be mounted in or on a holder that is arranged between the upper-part connection and the lower-part connection, in particular between the upper part and the lower part, wherein the holder itself can be of a multi-part design and has at least two degrees of freedom, in order to avoid jamming and tilting. The two degrees of freedom are preferably rotational degrees of freedom, wherein the rotation axes of the two degrees of freedom are perpendicular to each other, so as not to influence movements relative to each other.

At least one degree of freedom, in particular a rotational degree of freedom, is provided via an elongate hole guide or a rail guide, in order to easily and safely maintain the associations of the components to each other. An exchangeable pin can be guided inside the elongate hole guide, such that, in the region of the elongate hole guide, the joint device can be separated into several components, so as to permit easy assembly and disassembly of the joint device and therefore also easy fitting of the orthosis. By arranging the site of separation inside the elongate hole guide, a considerable range of travel is made available within which assembly can take place. Thus, the components of the joint device do not need to be aligned exactly with each other for assembly.

The invention likewise relates to an orthosis having an upper part and a lower part, which are connected to each other via a joint device, as described above.

Illustrative embodiments of the invention are explained in more detail below with reference to the accompanying figures, in which:

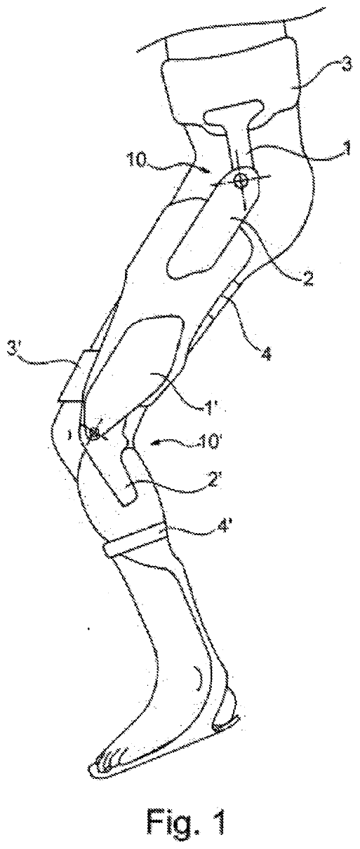

FIG. 1 shows a schematic overall view of an orthosis;

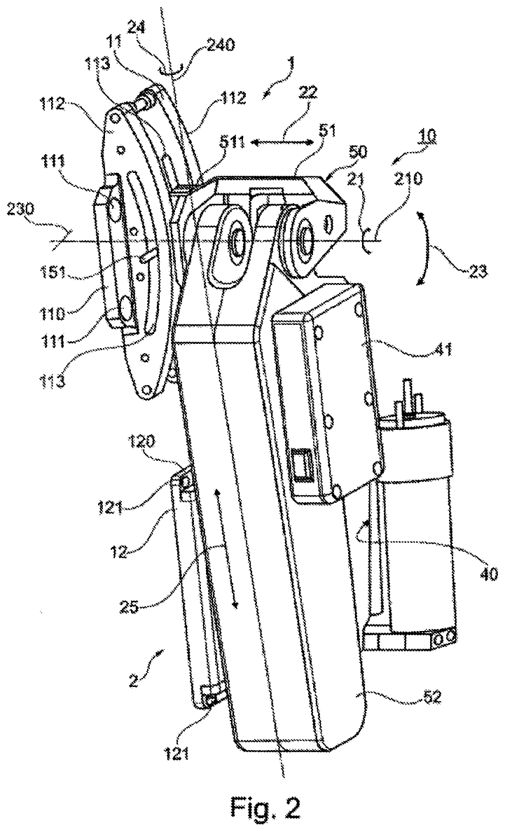

FIG. 2 shows a perspective view of a joint device without fastening device;

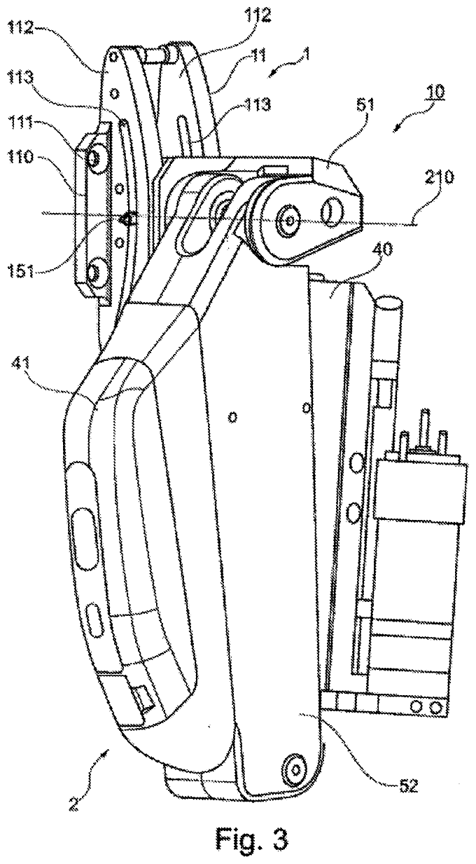

FIG. 3 shows a variant of FIG. 2, with a modified control appliance;

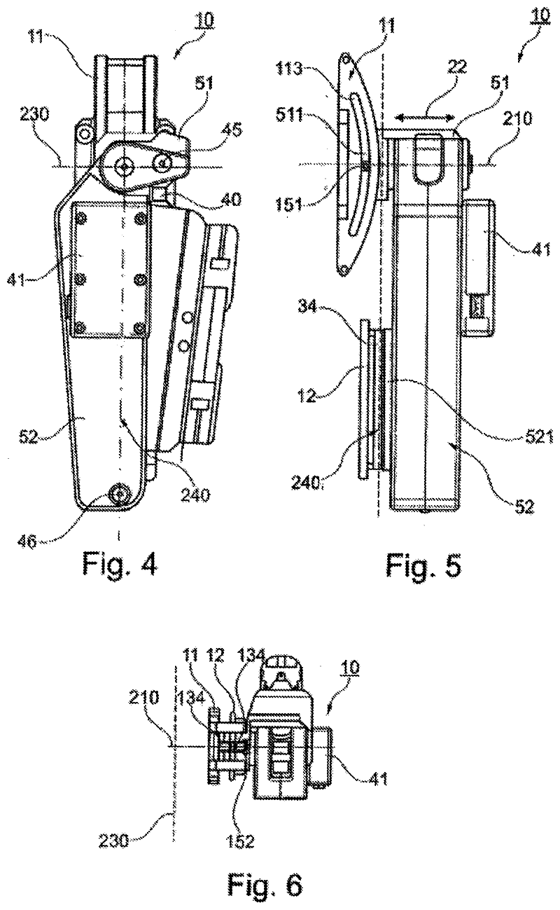

FIG. 4 shows a side view of the embodiment according to FIG. 2;

FIG. 5 shows a front view of FIG. 4;

FIG. 6 shows a plan view according to FIG. 5;

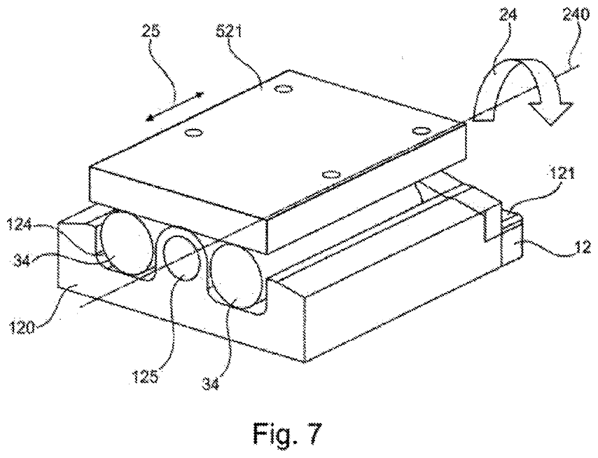

FIG. 7 shows a lower-part connection on its own;

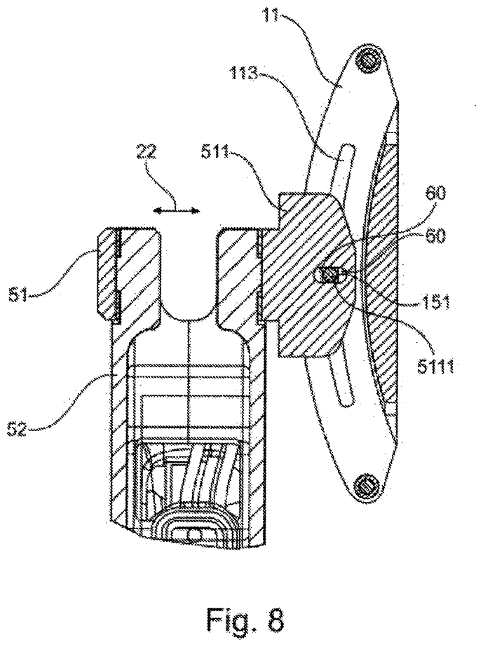

FIG. 8 shows a sectional view through an upper-part connection, and

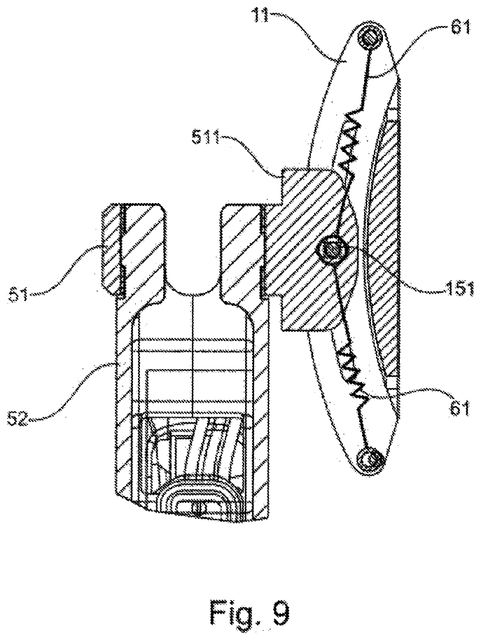

FIG. 9 shows a variant of FIG. 8.

FIG. 1 is a schematic overall view of a hip-knee-ankle orthosis which engages over three joints of the lower extremity, namely the hip joint, the knee joint and the ankle joint. In the region of the natural hip joint and in the region of the natural knee joint, a respective joint device 10, 10′ is arranged between an upper part 1, 1′ and a lower part 2, 2′, respectively. The respective upper part 1, 1′ is fitted on the patient via a first fastening device 3, 3′, and the respective lower part 2, 2′ is fastened, via a second fastening device 4, 4′, to the limb located distally with respect to the respective joint device 10, 10′. The orthosis thus has two upper parts 1, 1′. The first upper part 1 is fastened to the trunk of the patient via a pelvic strap or a hip strap 3. The associated lower part 2 is fastened to the upper leg via an upper-leg shell or upper-leg rail and a fastening strap as fastening device 4. The upper-leg shell together with the fastening device 4 at the same time forms a second upper part 1′, which is secured to the upper leg via a first fastening device 3′ provided for this purpose. Of course, the upper-leg shell is moreover connected to the upper leg moreover with the second fastening device 4 to the first upper part 1. A lower-leg rail is arranged as second lower part 2′ distally with respect to the upper-leg shell or upper-leg rail, which second lower part 2′ can be secured to the lower leg via a strap as second fastening device 4′. The lower-leg rail has a foot plate on which the foot is supported. The upper-leg shell with the associated fastening devices is thus both first lower part 2 and second upper part 1′ in the use as an orthosis provided with two joint devices 10, 10′.

FIG. 2 shows a perspective view of a joint device 10 as part of an orthosis which can be arranged on a patient so as to engage over the joints. The joint device 10 has an upper part 1, above a first rotation axis 210, and a lower part 2, wherein the upper part 1 has an upper-part connection 11 for securing to a first fastening device (not shown) on the pelvis of a patient. The lower part 2 has a lower-part connection 12 which can be fastened to a second fastening device in order to secure the lower part 2 to an upper leg. The upper-part connection 11 is provided with a base plate 110 in which bores 111 are arranged through which the upper-part connection 11 can be secured to the first fastening device. The securing can be effected via screws, for example. Two walls 112 extend from the base plate 110, wherein the two walls 112 are oriented substantially parallel to each other and are aligned perpendicularly with respect to the base plate 110. Inside each wall 112, an elongate hole guide 113 is formed which is designed as a partial arc of a circle or at least curved, wherein the radius of the elongate hole guide 113 is such that the midpoint lies outside the upper-part connection 11.

A sliding pin 151 is guided inside the elongate hole guide 113 and connects the upper-part connection 11 to a holder 50, which is of a multi-part design. The sliding pin 151 connects the holder 50 to the upper-part connection 11 reversibly. For this purpose, the sliding pin 151 is insertable into the elongate hole guide 113 and removable. The sliding pin 151 is guided through the elongate hole guide 113 and through a bore or an elongate hole 5111 in a web 511. The web 511 is part of a frame 51, which is in turn part of the holder 50. The frame 51 is substantially U-shaped and forms a rotary bearing via its side branches, such that a housing 52, which is a second part of the holder 50, is mounted rotatably about the rotation axis 210. The rotation axis 210 is substantially perpendicular to the sagittal plane and permits a pivoting movement of the lower part 2 inside the sagittal plane, such that a leg can swing forward and backward when an orthosis is fitted on the hip or the pelvis.

The housing 52, as part of the lower part 2, serves to receive an actuator 40, which can be configured as a damper and/or drive. The housing 52 can be part of an actuator housing or can consist entirely of the actuator housing. Laterally, i.e. on that side of the housing 52 directed away from the upper-part connection 11, a control device 41 is arranged which is coupled to sensors (not shown) and evaluates sensor data recorded by sensors and conveys these to an adjustment device or a motor control appliance, such that either damper properties are changed or a drive is activated or deactivated.

Distally with respect to the upper-part connection 11, a lower-part connection 12 with a base plate 120 and with bores 121 arranged in the latter is arranged on the medial side of the housing 52 directed toward the patient. By way of the base plate 120 and the bores 121, the lower-part connection 12 is secured to the second fastening device (not shown), such that the lower part 2 can be secured to the upper leg of the patient during use of the joint device as a hip joint.

It is possible via the joint device 10 to pivot the frame 51 about a rotation axis 230, since the frame 51 is guided inside the elongate hole guide 113 along a portion of an arc of a circle. The rotation axis 230 lies outside the joint device 10. The radius of the elongate hole guides 113 is preferably chosen such that the rotation axis 230 lies inside the hip joint, preferably in the anatomical hip rotation point.

The housing 52 and thus the entire lower part 2 is mounted pivotably on the frame 51 about the rotation axis 210.

In addition, the lower part 2 is arranged in the frame 51 through an elongate hole guide 5111 so as to be displaceable along the rotation axis 210 in the lateral and medial directions, as is indicated by the double 22, which represents a translational degree of arrow freedom. The displaceability along the rotation axis 210, i.e. in the medial-lateral direction, is limited by end stops, for example by the elastic buffer elements 60 shown in FIG. 8, such that only a limited displaceability within the side branches of the frame 51 is permitted. As an alternative to a displaceability between the side branches, the displaceability can be effected by a resilient mounting of the web 511 between the walls 112 by means of an elastic mounting of the sliding pin 151. Alternatively, the lower-part connection 12 can be mounted on the housing 52 so as to be longitudinally displaceable along the longitudinal extent of the housing 52, i.e. in the proximal-distal direction of the lower leg, as is indicated by the double arrow 25, which represents a translational degree of freedom. Here too, end stops are able to limit the displaceability of the lower-leg connection 12 relative to the housing 52. The lower-part connection 12 is additionally mounted pivotably about a rotation axis 240 which runs in the proximal-distal direction, i.e. in the longitudinal extent of the housing 52, as will be explained further below. Thus, the joint device 1 has a total of three rotational degrees of freedom 21, 23 and 24, which permit a rotatability of the components relative to one another about the rotation axis 210, the rotation axis 230 and the rotation axis 240. In addition, a translational degree of freedom 22 is present within the joint device 1, namely in the medial-lateral direction, by the displaceability within the side branches of the frame 51.

FIG. 3 shows a variant of the joint device 10 with the same mechanical set-up but with a control device 41 arranged at the front, by means of which it is possible for the joint device 10 to be designed narrower in the medial-lateral direction than in the variant according to FIG. 2. It will be seen in FIG. 3 that the sliding pin 151, which connects the frame 51 to the upper-part connection 11, lies at the height of the rotation axis 210, such that the rotation axes 230 and 210 intersect each other at one point.

FIG. 4 shows a side view of the joint device 10 according to FIG. 2, in which the orientation of the rotation axes 230 and 240 at right angles to each other can be seen. The figure also shows the actuator 40, which has an upper articulation point 45 spaced apart from the rotation axis 210. In the embodiment shown, a lower articulation point 46 of the actuator 40 in the housing 52 of the lower part 2 is located in alignment with the rotation axis 240 of the lower-part connection 12. By a pivoting about the rotation axis 240 along the rotational degree of freedom 21, the distance of the upper articulation point 45 from the lower articulation point 46 changes, such that the actuator, for example as a hydraulic damper, experiences a relative movement between piston and cylinder in a longitudinal direction. In the case of a damper, a resistance to the relative movement is provided by corresponding hydraulic resistances. For a drive, the actuator 40 is supplied externally with energy and, by means of a motor, causes the two articulation points 45, 46 to move toward each other or away from each other.

FIG. 5 shows a front view of the joint device 10 with the laterally arranged control device 41, and with the upper-part connection 11 arranged medially with respect to the housing 52 and the frame 51. The web 511 is fitted between the walls 112 of the upper-part connection 11 and is held by the sliding pin 151. The frame 51 can be displaced relative to the upper-part connection 11 along the degree of freedom 22. Alternatively, the housing 52 can be displaced inside the frame 51 along the rotation axis 210 in the medial-lateral direction, in order to form the translational degree of freedom 22.

Medially on the housing 52, the lower-part connection 12 is arranged pivotably about a rotation axis 240. Buffer elements 34, which are arranged as cord-shaped elastomer elements parallel to the rotation axis 240, serve on the one hand as an end stop and on the other hand as a restoring element, so as not only to form the rotational degree of freedom 24 about the rotation axis 240 but also to restore to the starting position shown. The starting position or the angle position of the end stops can be defined via the geometric configuration of the buffer elements 34, e.g. via their diameter. In addition, the elastic behavior about this degree of freedom can be influenced by the geometry and the material properties of the buffer elements 34.

FIG. 6 is a plan view of the joint device according to FIG. 5, showing that the rotation axis 230, about which pivoting is possible through the elongate hole guides 113, lies laterally with respect to the upper-part connection 11 and thus outside the joint device 10. The rotation axis 210 is perpendicular to the rotation axis 230.

FIG. 7 shows a detail of the lower-part connection 12 with the bores 121 for securing to the fastening device (not shown). Inside the base plate 120, recesses 124 are formed in the shape of channels, between which a bore or sleeve 125 is arranged. Inside the bore 125 runs the rotation axis 240, about which the lower-part connection 12 can be pivoted relative to a fastening plate 521 which can be secured to the housing 52 of the lower part 2 via the illustrated bores and screws. The fastening plate 521 can also be seen in FIG. 5.

The joint device 10 is kinematically indeterminate as a result of the configuration with at least four degrees of freedom; the orthosis system becomes kinematically determinate only when fitted to the user. In the example shown, the rotation axis 210 normal to the sagittal plane is automatically positioned through the physiological joint, in this s case the hip joint. Despite the positioning of the joint device 10 outside the body, the rotation axis 230 runs normal to the frontal plane through the physiological rotation point, which avoids constraints and resulting longitudinal loads. The exact determination of the anatomical rotation point of the natural hip joint is extremely difficult. By means of a joint device 10 as described above, an exact positioning is no longer necessary with the orthosis applied rigidly to the human body, since an automatic positioning over the physiological rotation point is effected on account of the available degrees of freedom.

By means of the removable sliding pin 151, it is possible for the joint device 10 to be designed to be easily assembled and disassembled in order to cancel and again restore the mutual association of the components of the joint device 10. In the illustrative embodiment, by the removal of the sliding pin 151, the upper-part connection 11 can be easily separated from the other components of the joint device 10 and secured thereon again, as a result of which it is considerably easier to apply the pelvic strap and separately apply, for example, an upper-leg rail, or an upper-leg rail with attached lower-leg rail and foot part. Specifically in the case of a hip-joint connection with an attached orthosis engaging over the knee joint and the ankle joint, assembly is otherwise very difficult, since both the ankle joint and the knee joint, and also the hip joint, have to be oriented within the orthosis, and the orthosis has to be secured to the leg. By canceling the association of the components and by being able to easily restore said association, the fitting of the orthosis can be made substantially easier.

The joint device 10 is stabilized in its basic position by the buffer elements 34. Passive-elastic properties can be defined by the choice of geometry and the material properties of the buffer elements 34. A shallow overall height is obtained, wherein the lower-part connection 12 can be designed laterally offset with respect to the upper-part connection 11, so as to be able to compensate for distance differences in the medial-lateral direction of the particular patient and in the mechanical configuration of the upper part 1 and of the lower part 2. With a shallow overall height in the medial-lateral direction, the joint device 10 permits great flexibility in terms of the adjustability of the desired neutral position and a high torsional stiffness. The neutral position and the torsional stiffness can be defined by the diameter, the contact length and the elastic properties of the respective buffer elements 34.

The change of the effective length of the buffer elements 34 can be easily realized in design terms by a suitable shaping and a possibility of displacement inside the channels 124, as a result of which the rotatability and the torsional stiffness about the rotation axis 240 can be adjusted easily and steplessly.

The functionality of the hip connection is achieved by means of the translational degrees of freedom in the sagittal plane being blocked. Moreover, the translational degree of freedom normal to the sagittal plane is in principle free, but limited by end stops. When the orthosis is fitted in place, this additional degree of freedom aligns the functional rotation point of the joint device 10 with the anatomical hip rotation point. The rotational degree of freedom in the frontal plane is passively stabilized. Through the adaptation of the restoring, passive elements or buffer elements 34, the stiffness can be optimally adjusted for the particular user. The possibility of movement normal to the sagittal plane, limited by the end stops 134 or the boundary of the elongate hole 5111, permits a deviation of the distance of the physiological rotation point from the radius predefined by the joint device 10, which radius derives from the radius of the elongate hole guide 113. This permits simple adaptation by the orthopedic technician. The separability of the upper-part connection from the remaining part of the joint device 10 is achieved by the sliding pin 151, which is guided through a stabilization nut 152 in the web 511, on which stabilization nut 152 elastic end-stop elements 134 engage, which counter a displacement in the medial-lateral direction.

The leg rotation is stabilized by the buffer elements 34 in the basic setting thereof and, on account of their structure, has great flexibility in terms of the desired torsional stiffness. The design of the joint device is such that the rotation is permitted with a very shallow compact structure, which has a very positive impact on the medial-lateral extent of the overall structure of the orthosis.

FIG. 8 shows a sectional view through an upper-part connection 11 with the arc-shaped elongate hole 113 which is formed therein and in which the web 511 is guided via the sliding pin 151. The frame 51 and the housing 52 (which is shown only in part) are fastened to the web 511 and coupled to the upper-part connection 11. Inside the web 511, an elongate hole 5111 is formed via which a medial-lateral movement according to the double arrow 22 is permitted. End stops or elastic buffer elements 60 can be arranged in the elongate hole 5111 in order, on the one hand, to permit an orientation in a desired zero setting and, on the other hand, to permit an elastic displaceability in the medial-lateral direction or in respect of the translational degree of freedom 22.

FIG. 9 shows a variant of FIG. 8, in which the sliding pin 151 is assigned elastic elements 61 which are functionally connected to the sliding pin 151. In this way, both the sliding pin 151 and the web 511 are held elastically in a zero setting inside the elongate hole 113. A displacement inside the elongate hole 113 is still possible. The elastic elements 61 can also limit the displacement movement inside the elongate hole 113 before the respective ends of the elongate hole 113 are reached. Instead of elastic elements 61, rigid bands, preferably of adjustable length, can also be assigned to the sliding pin 151, in order to ensure a stop limitation and an adjustment of the pivoting angle.

1. A joint device of an orthosis, comprising:
an upper part;
a lower part connected to the upper part in an articulated manner, wherein the joint device has at least four degrees of freedom between the upper part and the lower part;
a first fastening device configured to secure the upper part to a patient;
a second fastening device configured to secure the lower part to a limb;
an upper-part connection and a lower-part connection, via which the upper part and the lower part can be secured to the fastening devices, wherein the upper-part connection is attached to the first fastening device and the lower-part connection is attached to the second fastening device; and
an actuator assigned to at least one rotational degree of freedom of the at least four degrees of freedom, wherein the actuator is mounted in or on a holder, which is arranged between the upper part and the lower part and the holder has at least two degrees of freedom of the at least four degrees of freedom.

2. The joint device as claimed in claim 1, wherein the joint device has three rotational degrees of freedom, and wherein three of the at least four degrees of freedom are the three rotational degrees of freedom.

3. The joint device as claimed in claim 1 wherein the joint device has at least one translational degree of freedom, and wherein one of the at least four degrees of freedom is the at least one translational degree of freedom.

4. The joint device as claimed in claim 2, further comprising rotation axes for at least two of the rotational degree of freedom wherein the rotation axes intersect each other.

5. The joint device as claimed claim 2, wherein a pivot axis of at least one rotational degree of freedom of the at least three rotational degrees of freedom lies outside the joint device.

6. The joint device as claimed in claim 1, wherein at least one degree of freedom of the at least four degrees of freedom is limited via end stops.

7. The joint device as claimed in claim 1, wherein at least one elastic buffer element is assigned to at least one degree of freedom of the at least four degrees of freedom.

8. The joint device as claimed in claim 7, wherein the at least one elastic buffer element is formed by elastomer elements arranged parallel to the rotation axis of the at least one degree of freedom.

9. The joint device as claimed in claim 7, wherein the at least one elastic buffer element comprises at least one cord-shaped elastomer element.

10. The joint device as claimed in claim 7, wherein the joint device is held by the at least one elastic buffer element in a starting position with respect to at least one degree of freedom of the at least four degrees of freedom.

11. (canceled)

12. (canceled)

13. The joint device as claimed in claim 1, wherein the at least two degrees of freedom of the at least four degrees of freedom are rotational degrees of freedom, and rotation axes associated with the rotational degrees of freedom of the at least four degrees of freedom are perpendicular to each other.

14. The joint device as claimed in claim 1, wherein at least one rotational degree of freedom of the at least two degrees of freedom of the at least four degrees of freedom is at least one of formed via an elongate hole guide or limited via bands.

15. An orthosis having an upper part and a lower part, which are connected to each other via a joint device as claimed in claim 1.

16. A joint device of an orthosis, comprising:
an upper part;
a lower part pivotally connected to the upper part, wherein the joint device has at least four degrees of freedom between the upper part and the lower part;
a first fastening device configured to secure the upper part to a patient;
a second fastening device configured to secure the lower part to a limb of the patient;
an upper-part connection and a lower-part connection, via which the upper part and the lower part can be secured to the fastening devices, wherein the upper-part connection is attached to the first fastening device and the lower-part connection is attached to the second fastening device; and
an actuator assigned to at least one rotational degree of freedom of the at least four degrees of freedom, wherein the actuator is mounted in or on a holder, which is arranged between the upper part and the lower part and the holder has at least two degrees of freedom of the at least four degrees of freedom.

17. The joint device as claimed in claim 16, wherein the at least four degrees of freedom include at least three rotational degrees of freedom.

18. The joint device as claimed in claim 16, wherein at least four degrees of freedom include at least one translational degree of freedom.

19. The joint device as claimed in claim 17, further comprising rotation axes for at least two of the rotational degrees of freedom of the at least three rotational degrees of freedom, wherein the rotation axes intersect each other.

20. The joint device as claimed claim 17, wherein a pivot axis of at least one rotational degree of freedom of the at least three rotational degrees of freedom lies outside the joint device.
